(19) United States
(12) Patent Application Publication
(10) Pub. No.: US 2025/0000370 A1
(43) Pub. Date: Jan. 2, 2025

(54) MultiSensorsScope Device and Applications

(71) Applicant: Shraga Rottem, Forest Hills, NY (US)

(72) Inventor: Shraga Rottem, Forest Hills, NY (US)

(21) Appl. No.: 18/703,644

(22) PCT Filed: Oct. 24, 2022
(86) PCT No.: PCT/US2022/047620

§ 371 Date: Apr. 22, 2024

Related U.S. Application Data
(60) Provisional application No. 63/270,886, filed on Oct. 22, 2021.

(51) Int. Cl. A61B 5/02; A61B 1/00; A61B 1/303; A61B 5/00; A61B 5/0205; A61B 5/026; A61B 5/145; A61B 10/04

(52) U.S. Cl. CPC A61B 5/02007; A61B 1/00097; A61B 5/0205; A61B 10/04; A61B 1/303; A61B 5/0261; A61B 5/14503; A61B 5/4343; A61B 5/4839; A61B 5/4848

(57) ABSTRACT

A device usable directly in tumors with surface/subsurface angiogenesis wherein the tumor may be in cavities in the body for studying the micro circulation in other diseases for diagnostic use and to timely select the per patient therapy of choice. The device comprises a probe portion, a visualization portion, and a portion for sampling or intervention and a control station.

### FIELD OF THE INVENTION

The present invention relates to biosensors and medical visualizing scopes analyzing required data about the surface and subsurface micro circulation to; a) screen and diagnose cancers and other diseases; b) guide and monitor timely personalized therapy of such diseases; and c) accelerate the development and testing of drugs for such disease.

### BACKGROUND OF THE INVENTION

The problem in oncology today is that therapy is available for many cancers but works better if the cancers are diagnosed early.

There is need for biosensors that could screen and diagnose cancers early and to guide and monitor timely personalized therapy.

The current noninvasive methods used to diagnose cancers and to evaluate the effect of therapy are DNA analysis in patient's blood, CT, MRI, PET/CT, MAMMOGRAPHY and various endoscopic methods. As to monitoring the effect of therapy, most of the times (except in blood cancers) this is based on CT, MRI, PET-CT, MAMMOGRAPHY at least three months after the new treatment is started. Most of the medical imaging methods are based on comparing the size and appearance of the tumors and metastases at a previous scan, with images obtained three months later.

However, the effect of the therapies starts at local microlevel a few weeks after starting at treatments. Ineffective treatment is missing the opportunity of timely replacement with another anti-tumor agent and results in tumor continuing to grow and metastases (hence shortening of life), exposes the patients to unnecessary side effects and increases the cost of the therapy. Moreover, certain local biomarkers may have a role at the development of tumor angiogenesis, and intertumoral levels being different from area adjacent to the tumor and the certain change of values occur when successful therapy induces normalization or cancer related microcirculation to normal vessels.

### SUMMARY OF THE INVENTION

Most of the tumors (except blood and a few others) require angiogenesis to grow more than 2-3 mm. The mechanism of blood vessel formation by angiogenesis is initiated by the spontaneous dividing of tumor cells due to a mutation. Angiogenic stimulators are then released by the tumor cells. These then travel to already established, nearby blood vessels and activates their endothelial cell receptors. This induces a release of proteolytic enzymes from the vasculature. These enzymes target a particular point on the blood vessel and cause a pore to form. This is the point from where the new blood vessel will grow. The reason tumor cells need a blood supply is because they cannot grow any more than 2-3 millimeters in diameter without an established blood supply which is equivalent to about 50-100 cells. The blood vessels created in this way are not the same as normal blood vessels. Frequently, they are less organized and leakier than normal vessels with organized vasculature in normal tissue contrasting with chaotic vasculature in tumors.

A device viewing these tumor micro-vessels is touching them with precision. The detection of the differences in the flow of these micro vessels vs flow in normal micro vessels eliminates the current risk of confusing between organ vessels and tumor vessels, which are communicating. This is a problem unless the sensor touches a tumor vessel directly or it is recognizing it in surface and subsurface areas thanks to typical differences in flow. The present invention recognizes disorganization which is typical to tumor angiogenesis. In addition, the present biosensor device measures the levels of substances in a tumor area vs surroundings, correlates this with data from a microflow sensor, levels of mutated miRNA, DNA and other data, to establish a precise per case time oriented compounded score. This prognostic score will be used for diagnostic purposes, to monitor benign lesions prone to become cancerous, to monitor effectiveness of therapy and provide timely indication about the need to change the current drug being used, etc. The timely indication reassuring that the best oncology drug is being used results from observation of the change in the angiogenesis flow ahead of later shrinkage of the tumor or as sign of necrosis of the tumor even if the size is not reduced.

The Components A to E in the present invention are necessary to get the info on the local micro changes, with the combination of components being dictated on per case base.

The cancers which benefit from this invention are surface and subsurface tumors where the biosensor device can view the lesions and touch the surface and subsurface micro vessels to have the certainty that the measured flow is tumor microflow and not organ microflow. Measuring the flow of such small vessels (many times under 0.5 mm) and the evaluation of associated abnormal values vs normal local micro circulation requires a device with the following components with combinations of modalities to be indicated by the type of tumor and on per patient base:

A preferred embodiment of the present invention is the treatment of human papillomavirus (HPV), a cause of Cancer of the Cervix, Oral, Rectal and Penile Cancer) and or CIN (Carcinoma in Situ). For example, CIN have three grades based on the type of abnormal cells found on the surface of the cervix. CIN are usually lesions caused by certain types of HPV and detected at PAP test, with case management feasible by coloscopy, cervical biopsy, immunochemistry, molecular genetics. However certain CINs 1-2 are not cancer and usually go away on their own without treatment, but sometimes it can become cancer and spread into nearby tissue.

HPV infection is a viral infection, and here are more than 100 varieties of HPV. Some types of HPV infection cause warts, and some can cause different types of cancer. Most HPV infections do not lead to cancer. However, some types of genital HPV can cause cancer of the lower part of the uterus that connects to the vagina (cervix). Other types of cancers, including cancers of the anus, penis, vagina, vulva and back of the throat (oropharyngeal), have been linked to HPV infection. (Source: Mayo Clinic https://www.mayoclinic.org/diseases-conditions/hpv-infection/symptoms-causes/syc-20351596).

Nine of ten Cancers of the Cervix are caused by HPV. However, not all the cases with HPV will become cancerous lesions. Those which will become cancer will exhibit micro angiogenic changes typical to cancers. Periodic monitoring of the micro flow of cases with HPV using the present invention (as in FIG. 1-2), will identify these cases at early stages and allow timely therapy.

Cytology doesn't allow the study of the values of microangiogenesis showing which cases is case prone to become cancer or not, while the invention does.

CIN exhibiting micro-flow changes typical to cancer of the cervix should be treated more aggressively than just pre-cancerous CINs not showing such changes in the flow. References related to cancers and HPV: 1-15

### BRIEF DESCRIPTION OF THE FIGURES

FIG. 1 shows the periodic monitoring of the micro flow of cases with HPV using the present invention as well as in the microcirculation in other diseases and to accelerate the development and testing of new drugs.

FIG. 2 shows a second type of the periodic monitoring of the micro flow of cases with HPV and other diseases and to accelerate development and testing of new drugs using the present invention.

FIG. 3 shows a table listing cancers to be analyzed and treated by the different embodiments of the present invention via direct visualization and contact with the local micro circulation.

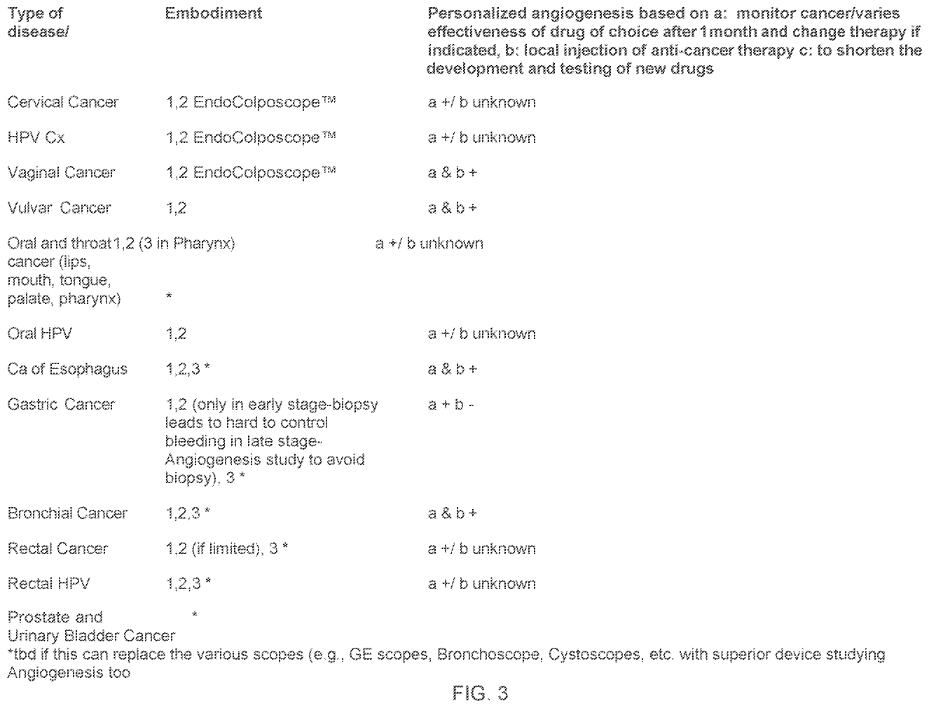

### DETAILED DESCRIPTION OF THE INVENTION

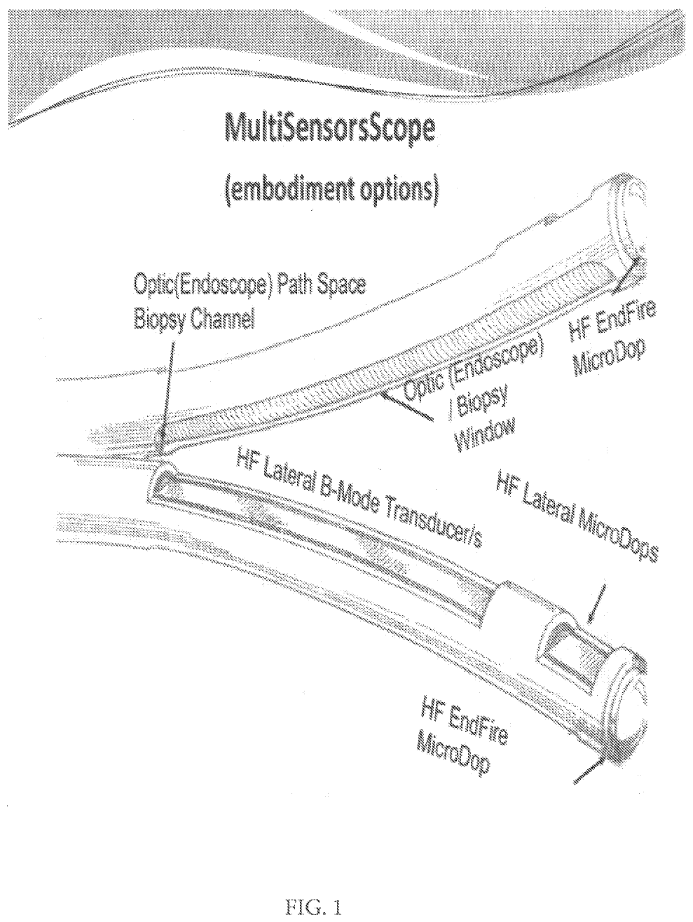

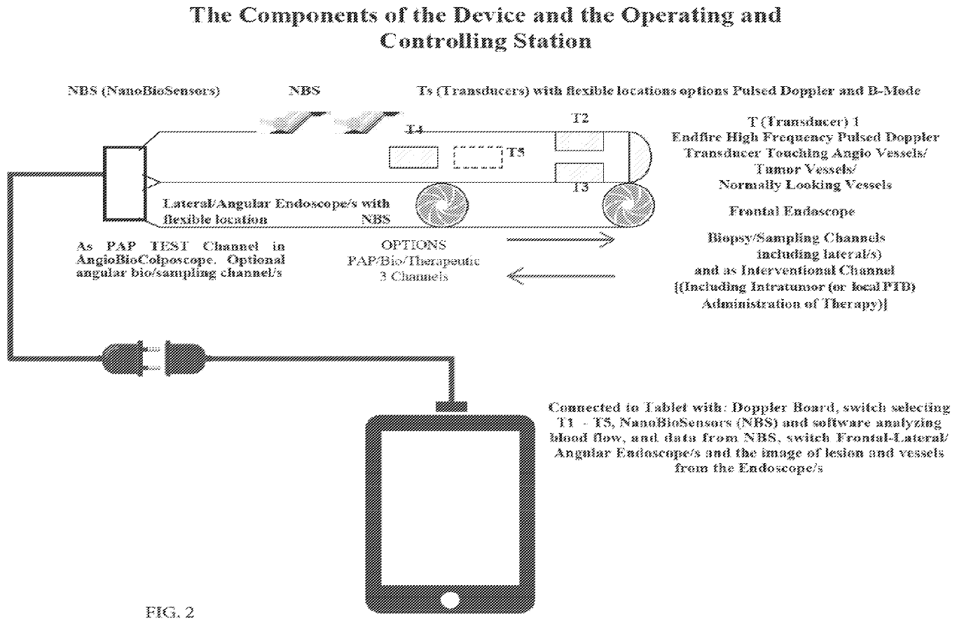

The components of the present embodiments of the invention are shown in the figures, with reference to the description. FIG. 1 with one type of embodiment, while FIG. 2 is a second embodiment, and the descriptions of the NanoBioSensors (NBS) use in Oncology, PTB (Preterm Labor), Pre-Eclampsia, PAD, IUI and Post Menopause.

Component A is high frequency multiplanar doppler transducers.

Tiny and most of the time submillimeter, high frequency pulsed doppler transducers are located as (a) endfire and (b) multiplanar to reach lateral micro vessels in cavities. The learning process of the values of such flow in angiogenesis vs normal communicating vessels are with special emphasis to surface or subsurface vessels, sometimes not deeper than 0.6 mm. The multiple doppler elements in the multiplanar configuration solve angle problems and also allow micro mapping of angiogenesis flow. A frequency of at least 20 MHz and size of maximum 1 mm is required to capture angiogenesis flow changes of such micro vessels (many times under 0.5 mm) and on surface or subsurface area up to 6 mm in depth. In certain cases, hematological migration of cancer cells to other anatomic areas (metastasis) can block flow of small capillaries in the areas, and be detected by the tiny high frequency pulsed doppler transducers.

While 6.5 MHz transvaginal probes are useful at detection of pelvic pathology such as uterine and ovarian tumors (Refs 10-14), they fall short from qualifying the flow pattern of vessels as benign or malignant, even when coupled with pulsed doppler. The micro communications between cancer and normal vessels and the cancer flow patterns can only be detected by a micro flow analyzing device with much higher doppler frequency touching the questionable micro vessels.

Component B encompasses multi-planar scopes. Tiny endoscopes are guiding and reassuring that the doppler elements are in fact touching angiogenesis vessels when they are visible, to assure correct angle of measurement via visualization of such angle or to guide that the measurement Is precisely done in the microlesion area. The max usability of the endoscopes is achieved by having a) frontal endoscope and b) multiplanar angular endoscopes.

Component C embodies the multiplanar sampling, Biopsy Therapeutic (SBT) Channel.

This channel is for simultaneous mini sampling in the correct area and adjacent areas for cytologic, genetic and biochemical values. This allows precise and efficient procedure to correlate values and data from components A and B, compared to doing a less precise sampling, which i) does not allow such precise correlation, ii) adds to risk of spreading metastases because of more invasive sampling to make sure there is enough tissue for this, as well as iii) complications associated with the need to repeat sampling or because of the need to access a tumor area to get precise data of angiogenesis and then again for sampling. The sampling channel can also be used for the purpose of precise timely local treatment when this is indicated as standalone or in combination with systemic chemotherapy, or immunotherapy etc. The SBT channel can also be used to seal vascular or tissue lesions.

Component D embodies (HF) high frequency multiplanar B-Mode transducers.

Since the angiogenesis flow may be in the subsurface area, adding High Frequency (HF) B-Mode transducers can guide the doppler sampling when the scopes cannot. The B-Mode transducers can be located next to multiplanar Doppler transducers, starting with one B-Mode transducer as exemplified in FIG. 1 and FIG. 2.

The embodiment of the components of the device will vary according with the clinical need. For example, in cases with advanced stomach cancer, a smaller device without SBT channel is indicated, otherwise these tumors can enter uncontrollable bleeding. In other applications (e.g., Angio Hysteroscope), the combination of all the components can precisely indicate the degree of the invasion of endometrial neoplasia, or guide precise multi biopsies and therapy such as polypectomy in case of endometrial polyps or adhesiolysis in case of Asherman's Syndrome.

Component E embodies the NanoBioSensors (NBS). The NanoBioSensors (NBS) are necessary to identify markers not detectable by changes in micro flow, to monitor the personalized anticancer drug concentration, which has a narrow therapeutic window, to allow for a personalized dosing of the drug correlated with the changes in angio values, improve the outcome and establish what is the required dose to achieve best results with minimum side effects. This is a necessary continuation of the other functions the MultiSensorScope™. The NBS can be located anywhere in the device and used each time when the device is used or implanted in the tumor via the SBT channels (the NBS which will record and transmit the data if implanted or transfer the data to the device via connection at a future procedure). When left in place, the NBS can include a nanobattery using the temperature of the human body to recharge. The NBS can be located or implanted via the lateral/angular sides of the device.

Components A,B,D,E will analyze a different type of data and signal when used in PTB (Preterm Birth) and Induction of Labor (see 6A and 6B) than in Oncology. Besides the embodiments as in the graphics in this application and in the implanted version, many type of graphics can be assembled as a band around the Uterine Cervix in early pregnancy and transmit data predicting PTB and of complications such as Pre Eclampsia along the way. The band configuration surrounding the uterine cervix will also capture and transmit data about mechanical changes in the cervix and collagen architecture. The usability of all the components (A,B,C,D,E) includes the drugs development and approval process as in 4B and in non-cancer drugs.

The present invention is not merely a combination of components in which each component functions in a known manner. As can be seen from the flow diagram of the figures and in the description of the device in the specification, the components of the present device have been chosen in a manner such that device achieves an overall interaction of components such that if one component cannot materially achieve a needed effect another component provides the lacking effect so that the overall device achieves the intended result.

### Additional Oncology Applicability of The New Device

A. The additional embodiment of this invention encompasses pointing at per patient benefit from oncology therapy and alternatives at early stage of the treatment.

In addition to the diagnostic role, the present invention also allows the measuring of the must have changes in the angiogenesis flow after e.g., a one month of chemo or immune therapy on per patient basis vs CT/MRI in three to four months. If the patients receive the appropriate drug, and guide the Oncologists as if to continue with the same therapy or combinations of therapies, or time change the therapy to improve the outcome and reduce unnecessary side effects and to lower the cost of the treatment. The timely indication reassuring that the best onco drug is being used, results from change in the angiogenesis flow ahead of later shrinkage of the tumor or as sign of necrosis of the tumor even if the size is not reduced. See also NBS usability.

B. The present invention further encompasses direct, early indicators of the effect (or the lack of effect) of a drug under development before and during FDA trials.

As an important part of the invention, the device will be used to observe the effect or no effect of new or improved drugs during development time which can be accomplished in e.g., one month thanks to the invention, instead of basing drug testing and approval process being based on the reduction of the size of tumor, which takes months, and many times wastes money just to realize that the drug is not worth the FDA process.

The device will indicate if the changes of angiogenesis flow (predicting later shrinking of tumor size) are present or absent. The timely indication that development of an oncology drug should proceed, results from change in the angiogenesis flow ahead of later shrinkage of the tumor or as angio sign of necrosis of the tumor even if the size is not reduced. The NBS are important because they can also indicate the necessary concentration of the therapeutic drug being tested, locally, and correlated with the angio signs.

The invention is crucial to speed the development of new therapies, with special emphasis to oncology drugs. It aims to save the lives of many patients waiting for such drugs to get FDA approval or having already metastatic disease by the time when the FDA approval of a drug is granted.

Getting FDA approval of a drug is a long and expensive process with cost going up to $2.6b and success rate around 12% of the applications to FDA and success rate of cancer drugs being much lower even though cancer drugs are 42% of all drug development programs.

One of the most important improvements of a parameter FDA seeks is evidence that there is a significant shrinkage of the tumors when approving certain Onco drugs.

The shrinking process requires changes in the flow of micro angiogenesis feeding solid tumors way before the tumor are found as shrinking at CT/MRI. The device in this invention provides this data and can be combined with other parameters mentioned in other claims. Data from the micro flow of tumor can be used as a standalone parameter or combined with other parameters (e.g., from NBS). The innovative device provides early direct evidence of a good reason to continue and accelerate the development of a cancer drug or to discontinue the development and save hundreds of millions of dollars in development and trials not going to show significant tumor shrinkage or signs anticipating necrotic process.

The use of the invention will accelerate the development of cancer drugs with millions of patients waiting for solution saving or extending their lives and becomes an urgent need.

Drugs development and testing Refs: 16-20

### Applicability in Tumors of Internal Organs Via Other Devices

C. The list of tumors that can benefit from this new Diagnostic Device is much bigger than the list of examples in Table 1, because the MultiSensorsScope can be in contact with tumors of internal organs (e.g., Liver and Pancreas and other organs) by introducing it e.g., via Laparoscope, Arthroscope, Spinal Scope or certain trocars. Depending on the access paths, and the number of components required, the size of the device can range from less than 1 mm to approximately 3 cm. TABLE 1 only shows examples of tumors accessible directly by the MultiSensorsScope without requiring insertion via other devices.

### Additional Details About the Use of The Device in Oncology

A. As an example, the present invention called the MultiSensorScope™ is required to view and precisely measure the microflow of Cervical Carcinoma, other Cervical Lesions, HPV, Vulvar Carcinoma and Vaginal Cancer, while also becoming the device for PAP test, hence replacing the Colposcope except for cases requiring major biopsy or tumor excision.

The MultiSensorScope (the present invention) is not limited to these surface and subsurface cancers. Without departing from the original invention, it can be used in other solid cancers with similar close to sensor distance, such as Oral Cancer and e.g., as novel Procto-Rectoscope, Hysteroscope, Esophagoscope, and other scopes, with part of these scopes being flexible rather than rigid and/or visualization and/or sampling parts reduced in size (see table of applications). Moreover, the sampling channel may be eliminated in the case of Gastroscope for the evaluation Cancer of the Stomach, to be limited to angiogenesis flow study under precise view/localization. Biopsy is not used in advanced stage of Gastric Ca because it can generate significant and hard to stop bleeding.

Similarly, when calculating the compounded score of microflow values and values of biomarkers in other cancers, the specific markers of each type of cancer will be used accordingly.

It is anticipated that a tumor may send metastases to other districts not developing into cancer but inducing changes in the micro circulation elsewhere or such distant micro circulatory changes are related to VEGF-Vascular Endothelial Growth Factor (e.g., a Gastro Intestinal cancer could show micro circulatory changes in the tongue without showing a tumor there). The new device also serves to look for such distant circulatory changes.

### Multi-Planar Transducers

Diagnosis based on sampling of multiple tumor vessels and score resulting from it makes the diagnosis safer compared with sampling of the flow of a single vessel and this includes subsurface areas which are not transparent enough to be visualized by the scopes, but detectable by HF B-Mode transducers. The degree of the departure of values of tumor flow from the flow of the normal vessels could give indication about personalized status and be even more precise if it becomes part of the score in A).

### Lateral/Angular Endoscopes

The frontal endoscope alone may not allow visualization of the lesion and vessels which are not in front of it, without time consuming, difficult maneuvers. Adding lateral/angular endoscope(s) allows a better view and shortens the time of the examination, especially in certain intra cavitary exams.

The lateral/angular endoscopes are needed to view the vessels to be touched and studied when they cannot be viewed via the frontal endoscope and sampled by the endfire transducer.

To improve the usability, the sampling channel should also have two angular accesses, like the transducers and scopes, in the same area +/−.

The device can be used to shorten the time of development of onco drugs by observing certain local changes starting with blood flow (compared with e.g., long term trial to show reduction of the size of the mass).

### The Hardware-Software Fusion

B. The micro angiogenesis data, data from normal vessels, endoscopic and sampling data (mentioned previously) will be combined with data from lab tests in three areas (tumor, surroundings, blood) and become a dedicated hardware-software fusion. It will help with personalized therapy in several types of cancers, including predicting risk of metastasis.

### REFERENCES

1-Nishida N, et al.: Angiogenesis in Cancer.
   https://pubmed.ncbi.nlm.nih.qov/17326328/
2. Nagy J. A et al.: Why are tumour blood vessels abnormal and why is it important to know?
   https://www.ncbi.nim.nih.qov./pmc/articles/PMC2661770/
3. Forster J. C. et al.: A review of the development of tumor vasculature and its effects on the tumor microenvironment
   https://www.ncbi.nlm.nih.qov/pmc/articles/PMC5395278/
4. Kein R. K.: Molecular regulation of vessel maturation
   https://pubmed.ncbi.nlm.nih.qov/12778167/
5. Elaine Y L Lu et al.: Human factors as a source of error in peak Doppler velocity measurement
   httpsJ/Pubmed.Ncbi.Nlm.Nih.Qov/16275456/
6. Mollica F, et al.: A model for temporal heterogeneities of tumor blood flow
   https://www.sciencedirect.com/science/article/pii/SO026286202000122?casa token=4n pY-nUpKdoAAAAA:ViprFXoeJt9KFktRgQoQzLWOPyNNWPnvIhodNWYbMXI-VaQYibJzqATM69ZORJSxXIPL9 tiIS6UQ
7. Ellis L M: Tumor Angiogenesis https-//www.sciencedirect.com/topics/medicine-and-dentistry/tumor-angioqenesis
8. Sieman D W: The Unique Characteristics of Tumor Vasculature and Preclinical Evidence for its Selective Disruption by Tumor-Vascular Disrupting Agentshttps://www.ncbi.nim.nih.qov/pmc/articles/PMC2958232/
9. Farmsworth R H et al.: Vascular remodeling in cancer
   https://www.nature.com/articies/onc2013304
10. Timor-Tritsch I, Baryam Y, Elgali S, Rottem S.: The technique of transvaginal Sonography with the use of a 6. 5 MHz probe Am J Obstet Gynecol 1988 May; 158(5):1019-24.https://pubmed.nobi.nlm.nih.gov/3285682/
11. Transvaginal Sonography. Timor Tritsch I and Rottem S. (Editors) Elsevier Sciences Publishing Co., New York 1988 First Edition
   https://www.abebooks.com/serviet/BookDetailsPL?bi=30845131918&cm sp=SEARCH REC--WIDGET-L--BDP-
   F&searchurl=an %3Dilan %2Btimor %2Btritsch %26sortby %3D17%26tn %3Dtransvaginal %2Bsonography
12. Transvaginal Sonography. Timor Tritsch I and Rottem S. (Editors) Elsevier Sciences Publishing Co., New York 1991 Second Edition https://www.amazon.com/Transvaginal-Sonoaraphy-Iian-Timor-Tritsch/dp/0444015779
13. Rottem S and Timor-Tritsch I: Ovary Pathology, in Transvaginal Sonography. Timor Tritsch I and Rottem S. (Editors) Elsevier Sciences Publishing Co., New York 1991 Second Edition, pp. 145-173 https://www.amazon.com/Transvaqinal-Sonoaraphy-Ilan-Timor-Tritsch/dp/0444015779
14. Rottem S., Timor-Tritsch I, Thaler I: Assessment of Pelvic Pathology Using High Frequency Transvaginal Sonography, in Ultrasound in Obstetrics and Gynecology, First Edition. Chervenak F A, Isaacson G C, Campbell S (Editors), Little, Brown and Company, Boston,1993.
   https://www.amazon.com/Ultrasound-Obstetrics-Gynecology-Frank-Chervenak/dp/0316138657
15. How Many Cancers Are Linked with HPV Each Year?
   https//www.cdc.gov/cancer/hpv/statistics/cases.htm
16. Phases of clinical trials
   https://www.cancer.net/research-and-advocacy/clinical-trials/phases-clinical-trials
17. A Tough Road: Cost To Develop One New Drug Is $2.6 Billion; Approval Rate for Drugs Entering Clinical Development is Less Than 12%
   https://www.policymed.com/2014/12/a-tough-road-cost-to-develop-one-new-drug-is-26-billion-approval-rate-for-drugs-entering-clinical-de.htmi
18. Wong C M et al.: Part 1: What Are the Chances of Getting a Cancer Drug Approved?MIT Computer Science & Artificial Intelligence Laboratory Department of Electrical Engineering & Computer Science
   https://qlobalforum.diaqlobal,org/issue/may-2019/what-are-the-chances-of-qetting-a-cancer-drug-approvedi
19. FDA approves third oncology drug that targets a key genetic driver of cancer, rather than a specific type of tumor
   https://www.fda,qov/news-events/press-announcements/fda-approves-third-oncology-drug-targets-key-genetic-driver-cancer-rather-specific-type-tumor
20. Modema's Keytruda combo misses in colorectal cancer as it shows promise in head and neck
   https://www.tiercebiotech.com/biotech/moderna-s-keytruda-combo-misses-colorectal-cancer-as-it-shows-promise-head-and-neck
21. Fighting Premature Birth: The Prematurity Campaign March of Dimes
   https://www.marchofdimes.org/mission/prematurity-campaign.aspx
22. Cost of Prematurity Birth-Analysis in New March of Dimes Report Card Shows Impact of Premature Birth
   https://unews.utah.edu/cost-of-preterm-birth/
23. Hedriana H, Byrne J, Campbell Bliss M, et al.: March of Dimes Preterm Labor Assessment Kit.
   https-/ohiohospitals.org/OHA,media/OHA-Media/Documents/Patient %20Safety %20and %200ualityInfant %20Mortality/EED %20W ebpaqe %20Resources/March-of-Dimes-Preterm-Toolkit.pdf
24. Moroz L. A. and Simshan N. N: Rate of sonographic cervical shortening and the risk of spontaneous preterm birth, Am J ObGyn 206;3, 2012
   https://www.sciencedirect.com/science/article/abs/pii/S0002937811023477
25. Amnisure
   https-//www.qiaqen.com/us/products/diagnostics-and-clinical-research/sexual-reproductive-health/maternal-fetaI-testin/amnisure-rom-test-10-min-us/
26. Parry S. et al.: Cervicovaginal fluid proteomic analysis to identify potential biomarkers for preterm birth, Am J Obstet Gynecol, 2020 May; 222(5):493.el-493.el3.
   https://pubmed.ncbi.nlm.nih.qov/31758918/
27. Feltovich H. and Berghella V: Beyond cervical length: emerging technologies for assessing the pregnant cervix, Am J ObGyn 207:5 2012
   https://www.sciencedirect.com/science/article/pii/S00029378120057892casa token=7ik Y5i7McB4AAAAA:3xz OsOYIcsLAmLIWY K9wBWyQkrKKDu9dgw3K5s91QOdigoO7M wSn8dXqpQfBIz4y EGdTZXCw
28. Bahos N.; Migliorelli F.; Posadas E.; Ferreri J et al.: Fetal Diagn Ther 2015; 38:161-169

### Definition of Failed Induction of Labor and Its Predictive Factors: Two Unsolved Issues of an Everyday Clinical Situation

https://www.karger.com/Article/Fulitext/433429
29. Selvin E. and Erlinger P.: Prevalence of and Risk Factors for Peripheral Arterial Disease in the United States; Circulation 2004
   https://www.ahaiournals.org/doi/tull/10.1161/01.CIR.0000137913.26087.F0
30. MacKenzie E., Jones A., Bosse M., Castillo R. et al.: Health-care costs associated with amputation or reconstruction of a limb-threatening injury, Journal of Bone and Joint Surgery, 2007
   https://www.safetylit.org/citations/index, php?fuseaction=citations.viewdetails&citationids (l=citjournalarticle 218354 38
31. Franklin H., Rajan M., Tseng C., Pogach L., et al.: Cost of lower-limb amputation in U.S. veterans with diabetes using health services data in fiscal years 2004 and 2010.

### J Rehabil Res Dev

https://www.rehab.research.va.gov/iour/2014/518/pdf/JRRD-2013-11-0249.pdf
32. Behroozian A. and Beckman J., Microvascular Disease Increases Amputation in Patients With Peripheral Artery Disease. Arteriosclerosis, Thrombosis, and Vascular Biology, 2020
   https://www.ahajournals.org/doi/10.1161/ATVBAHA.119.312859

### HPV:

Mayo Clinic—https//www.mayoclinic.org/diseases-conditions/hpv-infection/symptoms-causes/syc-20351596

### CIN-1:

NIH, National Cancer Institute, NCI Dictionaries-https//www.cancer.gov/publications/dictionaries/cancer-terms/def/cin-1

What is claimed:

1. Device usable directly in tumors with surface/subsurface angiogenesis wherein the tumor may be located in cavities in the body for studying the micro circulation in other diseases for diagnostic use and to timely select the per patient therapy of choice wherein the device comprises a probe portion a visionalization portion a portion for sampling or intervention and a control station.

2. The device according to claim 1 wherein surface/subsurface areas are not reachable directly by the device because the tumors are in internal organs, wherein any combination of elements in an embodiment can be used to reach such areas via Laparoscope, trocars etc.

3. The device according to claim 1 wherein in addition to the diagnostic use of the device and the use for the timely decision of per case based best therapy, the device is used in any embodiment to shorten the time required to develop and test new drugs.

4. The device according to claim 1 wherein in addition to the diagnostic use of the device related to the study of the microcirculation, the device also allows measuring the biochemical markers in the tumor vs peritumoral area and correlating this with microcirculation values and ct-DNA/miRNA and other case related bio markers.

5. The device according with claim 4 wherein a weighted combination of the per patient values is used to predict a best personalized therapy for a patient.

6. The device according with claim 1 wherein in addition the diagnostic use, the device is also used to deliver local anti-tumor (e.g., Chemotherapy) or anti prematurity treatment.

7. The device according with claim 5 wherein a weighted combination of per patient values is used to deliver best personalized therapy. E.g., in cancer, the weighted combination of values will be used to decide which case is suitable for local anticancer therapy instead of systemic (to reduce toxicity/side effects), which case is suitable for a mixture of local and systemic therapy and which case requires systemic therapy as main treatment.

8. The device according to claim 1 wherein in addition to the diagnostic use of the device related to the study of the microcirculation, the device also allows to detection of changes in the cervical microcirculation associated with preterm labor.

9. The device according to claim 1 wherein in addition to the diagnostic use of the device related to the study of the microcirculation, the device can monitor changes or lack of changes in the cervical microcirculation indicating correct choice of drug delaying labor or need to delay labor using a different drug.

10. The device according to claim 1 wherein in addition to the diagnostic use of the device related to the study of the microcirculation, the device can monitor changes or lack of changes in the cervical microcirculation indicating correct choice of drug delaying labor or need to delay labor using a different drug related to trial to get FDA for drugs delaying labor.

11. The device according to claim 1 wherein in addition to the diagnostic use of the device related to the study of the microcirculation, the device can detect changes associated with successful induction of labor after such induction is started.

12. The device according to claim 1 wherein in addition to the diagnostic use of the device related to the study of the microcirculation the device can detect changes in the cervical microcirculation associated with failed induction of labor after such induction is started and best alternative drug.

13. The device according to claim 1 wherein in addition to the diagnostic use of the device related to the study of the microcirculation, the device can detect changes in the cervical microflow and correlates these with hormonal changes in therapy of infertility including IVF.

14. The device according to claim 1 wherein in addition to the diagnostic use of the device related to the study of the microcirculation, the device can detect changes in the cervical microflow and corelate this with hormonal changes in therapy of post menopause.

15. The device according to claim 1 wherein in addition to the diagnostic use of the device related to the study of the microcirculation, the device can detect changes in the microflow in body areas at risk for amputation.

16. The device according to claim 1 wherein in addition to the diagnostic use of the device related to the study of the microcirculation, the device can monitor the effectiveness of new drugs aiming to delay amputation time, during the FDA trials

17. The device according to claim 1 wherein in addition to the diagnostic use of the device related to the study of the microcirculation, the device can be assembled as a band around the Uterine Cervix in early pregnancy and transmit data predicting PTB and of complications such as Pre-Eclampsia along the way.
